(19) United States
(12) Patent Application Publication
(10) Pub. No.: US 2021/0071123 A1
(43) Pub. Date: Mar. 11, 2021

(54) Inflatable Bioreactor and Method of Use

(71) Applicant: Global Life Sciences Solutions USA LLC, Marlborough, MA (US)

(72) Inventors: HannaLeena Saukkonen, Marlborough, MA (US); Ralph Stankowski, Westborough, MA (US)

(21) Appl. No.: 17/046,225

(22) PCT Filed: Apr. 25, 2019
(86) PCT No.: PCT/US2019/029143

§ 371 Date: Oct. 8, 2020

Related U.S. Application Data
(60) Provisional application No. 62/662,292, filed on Apr. 25, 2018.

(51) Int. Cl. C12M 1/00; C12M 3/06

(52) U.S. Cl. CPC C12M 23/14; C12M 27/20; C12M 27/16

(57) ABSTRACT

An inflatable bioreactor (110, 210) may include one or more sheets joined to form a bag (111, 211) including a top sheet (118, 218) and a bottom sheet (119, 219) formed from the one or more sheets and being inflatable to provide an internal volume (117, 217) suitable for retaining a volume of culture liquid (10) during flow of the culture liquid resulting from a rocking motion (R) of the bag, and one or more perturbing protrusions (116, 116′, 116″, 216) extending upwardly from the bottom sheet toward, but not as far as, the top sheet and extending transversely to, or obliquely to, a direction of wave motion (W) of the culture liquid. The resulting construction may provide improved flow for low initial volumes of the culture liquid in the bag.

### CROSS-REFERENCE TO RELATED APPLICATIONS

This application claims the benefit of U.S. Provisional Application No. 62/662,292, filed Apr. 25, 2018, the entire contents of which is incorporated by reference herein.

### TECHNICAL FIELD

The present disclosure relates generally to improvements in flexible bioreactors formed from a flexible sheet material, for example of the generally self-supporting type constructed as a bag or bag-like container for fluids for cultivation of cells or other biological material, and more particularly to inflatable flexible bag bioreactors for such cultivation under agitation, for example rocking. The disclosure also relates to improved bioreactor assemblies.

### BACKGROUND

The bio-processing industry has traditionally used stainless steel systems and piping in manufacturing processes for fermentation and cell culture. These devices are designed to be steam sterilized and reused. Cleaning and sterilization are however costly labour-intensive operations. Moreover, the installed cost of these traditional systems with the requisite piping and utilities is often prohibitive. Furthermore, these systems are typically designed for a specific process and cannot be easily reconfigured for new applications. These limitations have led to adoption of a new approach in recent years—that of using plastic, single-use disposable bags and tubing to replace the usual stainless steel tanks.

In particular bioreactors, traditionally made of stainless steel, have been replaced in many applications by disposable bags which are rocked to provide the aeration and mixing necessary for cell culture. These single-use bags are typically provided sterile and eliminate the costly and time-consuming steps of cleaning and sterilization. The bags are designed to maintain a sterile environment during operation, thereby minimizing the risk of contamination.

Commonly used bags are of the “pillow style,” mainly because these bags can be manufactured at low cost by seaming together two flexible sheets of plastic. Three-dimensional bags have also been described, where further sheets may be used to create wall structures.

Certain disposable bioreactor systems use a rocking table on to which a bioreactor bag is placed. The bioreactor bag is partially filled with liquid nutrient media and the desired cells. The table rocks the bag to provide constant movement of the cells in the bag and also efficient gas exchange from the turbulent air-liquid surface. The bag typically has at least one gas supply tube for the introduction of air, carbon dioxide, nitrogen, or oxygen, and at least one exhaust gas tube to allow for the removal of respired gases. Nutrients can be added through other tubes.

When cells are cultured at initial low volumes, mixing and oxygenation at those initial low volumes needs to be considered. Bags with baffles along the edges of the bag to improve the mixing have been described in U.S. Publication No. 2010/0203624 and U.S. Pat. No. 719,394, but these designs are not sufficient to effectively mix small volumes. Accordingly, there is a need for improved oxygenation in rocking table bioreactors for low volume cultures.

International Publication No. WO 2012/128703 addresses the need for better oxygenation by means of baffles extending vertically in a bag, but the described design does not address the need for improved oxygenation at low initial volumes.

A need therefore remains for improved bioreactor bags and bioreactor systems for cultivation of cells or other biological material, which address one or more of the above-described limitations of existing technology and are able to be used to provide necessary oxygenation for initial low volume cultures.

### SUMMARY

The present disclosure provides improved bioreactors and bioreactor systems for use in the cultivation of cells or other biological material. According to one aspect, an inflatable bioreactor is provided. In one embodiment, the bioreactor may include one or more sheets joined to form a bag including a top sheet and a bottom sheet formed from the one or more sheets and being inflatable to provide an internal volume suitable for retaining a volume of culture liquid during flow of the culture liquid resulting from a rocking motion of the bag. The bioreactor also may include one or more perturbing protrusions extending upwardly from the bottom sheet toward, but not as far as, the top sheet and extending transversely to, or obliquely to, a direction of wave motion of the culture liquid.

In certain embodiments, the one or more perturbing protrusions may be heat-sealed to the bottom sheet. In certain embodiments, the one or more perturbing protrusions may extend linearly in a direction transverse to or oblique to the direction of wave motion. In certain embodiments, the bag may include a first end edge and a second end edge positioned opposite one another, and a first side edge and a second side edge positioned opposite one another. In certain embodiments, the one or more perturbing protrusions may be positioned centrally between the first end edge and the second end edge. In certain embodiments, the one or more perturbing protrusions may have a vertical dimension that is equal to or less than about ¼ of a height of the fully inflated bag at a point above the one or more perturbing protrusions. In certain embodiments, the one or more perturbing protrusions may have a horizontal dimension that is equal to or greater than about ½ of a distance between the first side edge and the second side edge. In certain embodiments, the one or more perturbing protrusions may include a first perturbing protrusion and a second perturbing protrusion spaced apart from one another in a direction from the first side edge to the second side edge to define a gap therebetween. In certain embodiments, the one or more perturbing protrusions may include a first perturbing protrusion and a second perturbing protrusion spaced apart from one another in a direction from the first end edge to the second end edge. In certain embodiments, the one or more perturbing protrusions may have an inverted T-shape. In certain embodiments, the one or more perturbing protrusions may include an internal chamber and a plurality of sparge holes for directing a gas into the internal volume of the bag. In certain embodiments, the sparge holes may be positioned on vertical surfaces of the one or more perturbing protrusions. In certain embodiments, the sparge holes may be positioned on horizontal surfaces of the one or more perturbing protrusions.

According to another aspect, a bioreactor system is provided. In one embodiment, the bioreactor system may include a tray suitable for supporting an inflatable bioreactor in a rocking motion. The bioreactor may include one or more sheets joined to form a bag including a top sheet and a bottom sheet formed from the one or more sheets and being inflatable to provide an internal volume suitable for retaining a volume of culture liquid during flow of the culture liquid resulting from the rocking motion of the bag. The bioreactor system further may include one or more perturbing protrusions extending upwardly from the bottom sheet toward, but not as far as, the top sheet and extending transversely to, or obliquely to, a direction of wave motion of the culture liquid. In certain embodiments, the one or more protrusions may include one or more upward extensions of the tray.

According to another aspect, an inflatable bioreactor is provided. The inflatable bioreactor may include one or more sheets joined to form a bag including a top sheet and a bottom sheet formed from the one or more sheets and being inflatable to provide an internal volume suitable for retaining a volume of culture liquid during flow of the culture liquid resulting from a rocking motion of the bag. The bioreactor further may include one or more sparge ports positioned at least partially within the bag and attached to the bottom sheet. The one or more sparge ports may be in fluid communication with the internal volume and configured for delivering a gas thereto.

In certain embodiments, the inflatable bioreactor also may include one or more inlet ports in fluid communication with the one or more sparge ports and configured for delivering the gas thereto. In certain embodiments, the inflatable bioreactor also may include one or more sparge channels in fluid communication with the one or more sparge ports and configured for delivering the gas thereto. The one or more sparge channels may be defined by one or more sheets attached to the bottom sheet. In certain embodiments, the one or more sparge channels may be positioned outside of the bag. In certain embodiments, the one or more sparge channels may be positioned within the bag.

These and other aspects and embodiments of the present disclosure will be apparent or will become apparent to one of ordinary skill in the art upon review of the following detailed description when taken in conjunction with the several drawings and the appended claims.

The present disclosure extends to any combination of components or features disclosed herein, whether or not such a combination is mentioned explicitly herein. Further, where two or more components or features are mentioned in combination, it is intended that such components or features may be claimed separately without extending the scope of the disclosure.

### BRIEF DESCRIPTION OF THE DRAWINGS

Embodiments of the present disclosure can be put into effect in numerous ways. In describing illustrative embodiments of the disclosure, reference is made to the accompanying drawings, which are not necessarily drawn to scale, and wherein:

FIG. 1 is a side view of a bioreactor assembly in accordance with one or more embodiments of the disclosure, showing a bioreactor, a tray, a pivot block, and an actuator;

FIG. 2 is a perspective view of a bioreactor bag in accordance with one or more embodiments of the disclosure, showing a bag, support rods, and a perturbing protrusion;

FIG. 3 is a perspective view of a perturbing protrusion in accordance with one or more embodiments of the disclosure;

FIG. 4 is a perspective view of a perturbing protrusion in accordance with one or more embodiments of the disclosure;

FIG. 5A is a top view of a bioreactor bag in accordance with one or more embodiments of the disclosure, showing a bag, support rods, and a perturbing protrusion;

FIG. 5B is a top view of a bioreactor bag in accordance with one or more embodiments of the disclosure, showing a bag, support rods, and perturbing protrusions;

FIG. 5C is a top view of a bioreactor bag in accordance with one or more embodiments of the disclosure, showing a bag, support rods, and perturbing protrusions;

FIG. 5D is a top view of a bioreactor bag in accordance with one or more embodiments of the disclosure, showing a bag, support rods, and perturbing protrusions;

FIG. 6 is a side cross-sectional view of a portion of a bioreactor assembly in accordance with one or more embodiments of the disclosure, showing respective portions of a bag of a bioreactor bag and a tray having a perturbing protrusion;

FIG. 7 is a perspective view of a bioreactor bag in accordance with one or more embodiments of the disclosure, showing a bag, support rods, and a perturbing protrusion having sparge holes;

FIG. 8A is a side cross-sectional view of a portion of a bioreactor bag in accordance with one or more embodiments of the disclosure, showing respective portions of a bag and a perturbing protrusion having sparge holes;

FIG. 8B is a side cross-sectional view of a portion of a bioreactor bag in accordance with one or more embodiments of the disclosure, showing respective portions of a bag and a perturbing protrusion having sparge holes;

FIG. 9 is a perspective view of a bioreactor bag in accordance with one or more embodiments of the disclosure, showing a bag, support rods, and sparge ports;

FIG. 10 is a side cross-sectional view of a portion of a bioreactor bag in accordance with one or more embodiments of the disclosure, showing respective portions of a bag and sparge ports;

FIG. 11 is a top view of a bioreactor bag in accordance with one or more embodiments of the disclosure, showing a bag, support rods, and sparge channels;

FIG. 12A is a side cross-sectional view of a portion of a bioreactor bag in accordance with one or more embodiments of the disclosure, showing respective portions of a bag and a sparge channel; and

FIG. 12B is a side cross-sectional view of a portion of a bioreactor bag in accordance with one or more embodiments of the disclosure, showing respective portions of a bag and a sparge channel.

### DETAILED DESCRIPTION

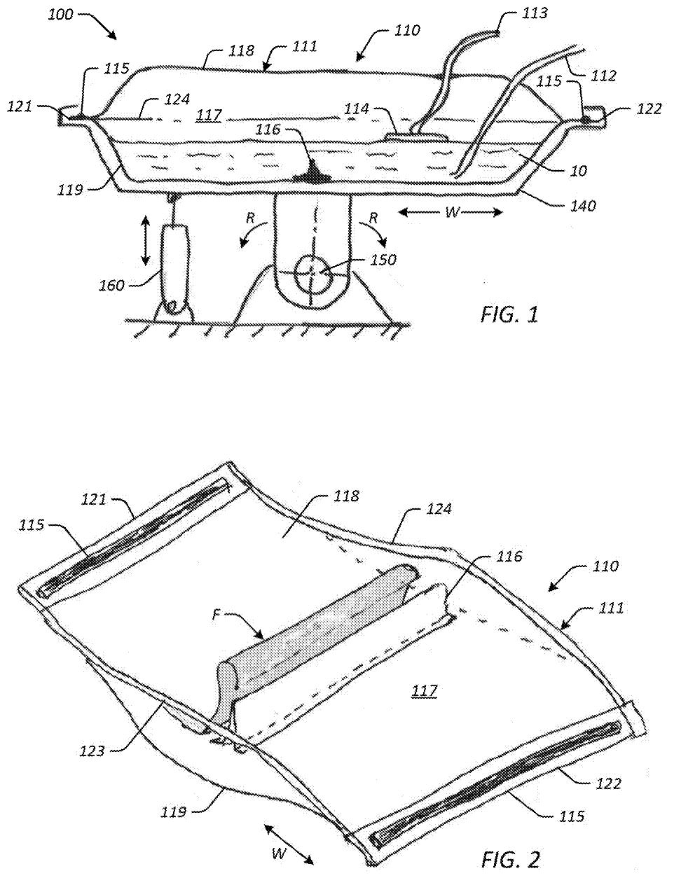

FIG. 1 illustrates a perfusion cell culture bioreactor system 100 (which also may be referred to herein as a “bioreactor system,” a “rockable bioreactor system,” or a “bioreactor assembly”) and components and features thereof according to one or more embodiments of the disclosure. The bioreactor system 100 may be used for cultivation of cells or other biological material, as described below. For example, the bioreactor system 100 may receive and agitate a cell culture medium 10 (which also may be referred to herein as a “culture liquid” or a “culture fluid”) to provide the aeration and mixing necessary for cell culture. As shown, the bioreactor system 100 may include a bioreactor 110, a tray 140, a pivot block 150, and an actuator 160. It will be appreciated that the bioreactor system 100 is illustrated schematically in FIG. 1, and that the bioreactor system 100 may include additional components and/or features according to various embodiments.

During operation of the bioreactor system 100, the bioreactor 110 may be supported by the tray 140, as shown in FIG. 1. The bioreactor 110 may be removably attached to the tray 140 via mating features thereof, as described below. When the bioreactor 110 is attached to the tray 140, the mating features may maintain the position and orientation of the bioreactor 110 relative to the tray 140. The pivot block 150 may be configured for pivotably supporting the tray 140, and the actuator 160 may be configured for inducing a back-and-forth rocking motion R (indicated by arrows) of the tray 140 and the bioreactor 110. During operation of the bioreactor system 100, the tray 140 may be caused to rock back and forth about the pivot block 150, under the influence of the actuator 160. Such rocking motion R may cause the cell culture medium 10 in the bioreactor 110 to flow in a wave-like motion W urged to the lowest part of the bioreactor 110 under the influence of gravity. Such wave motion W of the cell culture medium 10 may mix the cells into the medium liquid and provide a constant replenishment of oxygen and nutrients required for cell division. In some embodiments, the bioreactor system 100 may include a cooling system configured to cool the cell culture medium 10, which may allow the system 100 to accommodate higher cell densities. For example, a cooling system including a Peltier plate or a plurality of cooling fluid pipes arranged in a circuit may be positioned on or incorporated in the tray 140 for cooling the cell culture medium 10 in the bioreactor 110. Such a system also may be used for heating the cell culture medium 10 for lower cell densities.

The bioreactor 110 (which also may be referred to herein as a “bioreactor bag,” an “inflatable bioreactor,” or a “rockable bioreactor”) may be configured for receiving and containing the cell culture medium 10 during operation of the bioreactor system 100. As shown in FIG. 1, the bioreactor 110 may include a bag 111, an inlet port 112, an outlet port 113, a filter 114, a pair of support rods 115, and a perturbing protrusion 116.

The bag 111 may be formed of a flexible sheet material and may be inflated during use of the bioreactor 110. When inflated, the bag 111 may have an internal volume 117 (which also may be referred to herein as a “culture volume”) in which the culture medium 10 and cells can be aseptically cultured. The bag 111 may be formed by one or more sheets of flexible material, such as a flexible plastic material. For example, the bag 111 may include a top sheet 118 and a bottom sheet 119 of flexible material joined together to form the bag 111, as shown in FIGS. 1 and 2. In some embodiments, the top sheet 118 and the bottom sheet 119 may be joined together by means of heat introduced sealing or welding along respective edges of the sheets 118, 119, although other suitable means of joining the sheets 118, 119 may be used. In some embodiments, as shown, the top sheet 118, the bottom sheet 119, and the overall bag 111, may have a generally rectangular shape when viewed from the top or the bottom of the bag 111. In some embodiments, the bag 111 may have a first end edge 121 and a second end edge 122 positioned opposite one another, and a first side edge 123 and a second side edge 124 positioned opposite one another. In some embodiments, the top sheet 118 and the bottom sheet 119 may be separately formed and joined together along each of the edges 121, 122, 123, 124, for example by heat sealing. In some embodiments, the top sheet 118 and the bottom sheet 119 may be formed from a single piece of flexible material by folding the material to form one of the edges 121, 122, 123, 124 and joining the sheets 118, 119 together along each of the remaining edges 121, 122, 123, 124, for example by heat sealing. In some embodiments, the bag 111 may include one or more additional sheets of flexible material, in addition to the top sheet 118 and the bottom sheet 119.

As shown in FIG. 1, the inlet port 112 and the outlet port 113 may be in fluid communication with the internal volume 117 of the bag 111. During use of the bioreactor 110, the cell culture medium 10 may be continuously exchanged via the inlet port 112 and the outlet port 113, while the cells remain suspended in the cell culture medium 10 within the bag 111. As shown, the inlet port 112 may be formed as a tubular member including an internal portion positioned within the bag 111 and an external portion positioned outside of the bag 111, although other configurations of the inlet port 112 may be used. During use of the bioreactor 110, nutrients and dissolved oxygen may be continuously added via the inlet port 112. As shown, the outlet port 113 also may be formed as a tubular member including an internal portion positioned within the bag 111 and an external portion positioned outside of the bag 111, although other configurations of the outlet port 113 may be used. As shown, the filter 114 may be positioned within the bag 111 and attached to the internal end of the outlet port 113. In this manner, the flow of cell culture medium 10 out of the bag 111 may pass through the filter 114 such that inhibiting or toxic low molecular waste products are removed while the cells are kept in the bag 111. In some embodiments, the filter 114 may be a microfilter that is configured such that any cultured proteins expressed by the cells can be recovered in the permeate by known techniques. In some embodiments, the filter 114 may be an ultrafilter that is configured to allow expressed proteins to remain in the cell suspension for recovery in a batch harvest operation.

As shown in FIGS. 1 and 2, the support rods 115 may be attached to the bag 111 and positioned at or near the respective ends of the bag 111. For example, one of the support rods 115 may be positioned at or near the first end edge 121, and the other support rod 115 may be positioned at or near the second end edge 122. The support rods 115 may facilitate removable attachment of the bioreactor 110 to the tray 140 and maintaining the position and orientation of the bioreactor 110 relative to the tray 140 during operation of the bioreactor system 100. For example, the support rods 115 may be received and retained within respective channels or other mating features of the tray, as shown in FIG. 1, when the bioreactor 110 is attached to the tray 140. In some embodiments, the support rods 115 may be formed of a rigid or substantially rigid material. In some embodiments, the support rods may be formed of a material that is more rigid than the material of the bag 111. In some embodiments, the support rods 115 may be positioned and captured between the top sheet 118 and the bottom sheet 119 of the bag 111. For example, the support rods 115 may be attached to the bag 111 via the heat seals formed along the first end edge 121 and the second end edge 122 of the bag 111, although other means of attaching the support rods 115 to the bag 111 may be used.

As shown in FIGS. 1 and 2, the perturbing protrusion 116 (which also may be referred to herein as a “turbulence protrusion” or a “turbulence rib”) may be positioned within the bag 111 at or adjacent the bottom of the bag 111. The perturbing protrusion 116 may be configured to provide necessary oxygenation for initial low volume cultures. In particular, during operation of the bioreactor system 100, the perturbing protrusion 116 may agitate small initial volumes of the cell culture medium 10, by causing a “weir” like effect when the volume of cell culture is small, but may have less effect when the volume of culture liquid is greater. This effect is illustrated in FIG. 2, where the bag 111 is shown including the top sheet 118 and the bottom sheet 119 of flexible plastics sheet material. As shown, the perturbing protrusion 116 may extend transversely to the direction of the wave motion W mentioned above, with the result that the wave of the cell culture medium 10 breaks-up, like a wave breaking on a beach, so that a turbulent, non-laminar flow is generated, each time the rocking motion R of the tray 130 is made. Such a turbulent flow F is schematically illustrated in FIG. 2 as moving in one direction over the perturbing protrusion 116, although it will be appreciated that the turbulent flow F will occur successively in both directions over the perturbing protrusion 116 due to the back-and-forth rocking motion R of the tray 140.

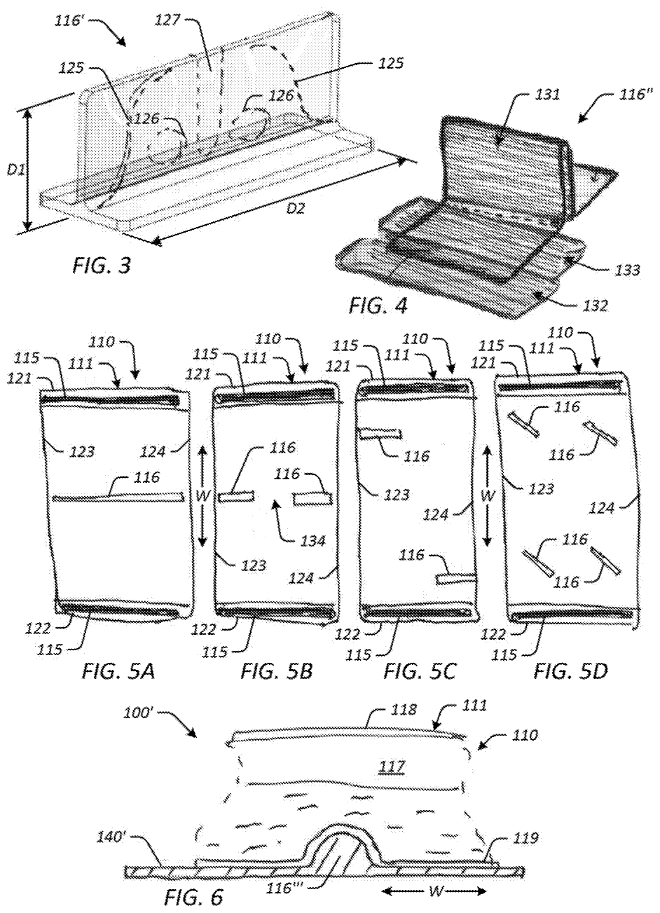

FIG. 3 illustrates a perturbing protrusion 116′ and features thereof according to one or more embodiments of the disclosure. The perturbing protrusion 116′ may be used in the bioreactor 110 in a manner similar to the perturbing protrusion 116 described above. As shown, the perturbing protrusion 116′ may be formed from an inverted T-shaped plastics extrusion, e.g., ethylene vinyl acetate (EVA) or low-density polyethylene (LDPE). Similar to the perturbing protrusion 116, the perturbing protrusion 116′ may be positioned within the bag 111 and heat-sealed to the bottom sheet 119 of the bag 111. As shown, the perturbing protrusion 116′ may have a vertical dimension D1 (which also may be referred to as a “height”) and a horizontal dimension D2 (which also may be referred to as a “length”). In some embodiments, the vertical dimension D1 may be equal to or less than about ¼ of the height of the fully inflated bag 111 at the point above the perturbing protrusion 116′. In some embodiments, the vertical dimension D1 may be about 1/10 of the height of the fully inflated bag 111 at the point above the perturbing protrusion 116′. In some embodiments, the horizontal dimension D2 may be equal to or greater than about ½ of the distance between the first side edge 123 and the second side edge 124 of the fully inflated bag 111. In some embodiments, the horizontal dimension D2 may be equal to or greater than about ¾ of the distance between the first side edge 123 and the second side edge 124 of the fully inflated bag 111. In some embodiments, the end profiles of the perturbing protrusion 116′ may be modified to be as shown by the chain-dotted lines 125, thereby to reduce sharp edges and to allow some fluids to flow around the perturbing protrusion 116′ when only very low volumes of culture medium and cells 10 are first introduced into the bag 111. In some embodiments, the perturbing protrusion 116′ may include one or more holes 126 and/or one or more cut-outs 127 to achieve a similar effect, allowing some fluids to flow through or between respective portions of the perturbing protrusion 116′.

FIG. 4 illustrates a perturbing protrusion 116″ and features thereof according to one or more embodiments of the disclosure. The perturbing protrusion 116″ may be used in the bioreactor 110 in a manner similar to the perturbing protrusion 116 described above. As shown, the perturbing protrusion 116′ may be formed from a plastics sheet of material which has resilience, folded into an inverted T-shape, and heat sealed together at a first heat seal 131 to maintain the inverted T-shape. The perturbing protrusion 116″ then may be fixed to the bottom sheet 119 of the bag 111 by further heat sealing at a second heat seal 132 and a third heat seal 133. Similar to the perturbing protrusion 116, the perturbing protrusion 116″ may be positioned within the bag 111 and fixed to the bottom sheet 119.

FIGS. 5A-5D illustrate examples of where one or more of the perturbing protrusions 116, perturbing protrusions 116′, or perturbing protrusions 116″ (collectively perturbing protrusion(s) 116) may be positioned and oriented relative to the bag 111 of the bioreactor 110 according to one or more embodiments of the disclosure. In FIG. 5A, a single perturbing protrusion 116 is positioned in the same location and orientation relative to the bag 111 as shown in FIG. 2. In particular, the perturbing protrusion 116 may be positioned generally centrally of the bag 111, midway between the first end edge 121 and the second end edge 122. As shown, the perturbing protrusion 116 may extend transversely to the direction of intended wave motion W in the bag 111 and across a majority of the distance between the first side edge 123 and the second side edge 124.

In some embodiments, as shown in FIG. 5B, the bioreactor 110 may include a pair of perturbing protrusions 116 positioned generally centrally of the bag 111, midway between the first end edge 121 and the second end edge 122. The perturbing protrusions 116 may be offset or spaced apart from one another in the direction between the first side edge 123 and the second side edge 124 to define a gap 134 between the perturbing protrusions 116. The gap 134 may allow very small volumes of liquids in the bag 111 to flow between the perturbing protrusions 116 in the direction of intended wave motion W in the bag 111. As shown, the perturbing protrusions 116 may extend transversely to the direction of intended wave motion W.

In some embodiments, as shown in FIG. 5C, the bioreactor 110 may include a pair of perturbing protrusions 116 offset or spaced apart from one another in the direction of intended wave motion Win the bag 111, again to allow flow of small volumes of liquids. In this manner, one of the perturbing protrusions 116 may be positioned closer to the first end edge 121, and the other perturbing protrusion 116 may be positioned closer to the second end edge 122. In some embodiments, the perturbing protrusions 116 also may be offset or spaced apart from one another in the direction of between the first side edge 123 and the second side edge 124. As shown, the perturbing protrusions 116 may extend transversely to the direction of intended wave motion Win the bag 111.

In some embodiments, as shown in FIG. 5D, the bioreactor 110 may include one or more perturbing protrusions 116 extending obliquely to the direction of intended wave motion W in the bag 111, such that an enhanced mixing action is introduced to the liquids in the bag 111. For example, the bioreactor 110 may include four perturbing protrusions 116 extending obliquely to the direction of intended wave motion W and offset or spaced apart from one another, as shown. Whilst the perturbing protrusions 116 illustrated in FIGS. 5A-5D are shown as extending linearly in plan view, in some embodiments, the perturbing protrusions 116 may be curved or may have an irregular shape, and have equal effect.

FIG. 6 illustrates a portion of a bioreactor system 100′ and components and features thereof according to one or more embodiments of the disclosure. Aside from the differences illustrated in FIG. 6 and described herein, the bioreactor system 100′ may be configured in a manner similar to the bioreactor system 100 described above, including similar components and features. As shown, the bioreactor system 100′ may include a tray 140′ having a perturbing protrusion 116′″ which in turn encroaches into the internal volume 117 normally provided by the bag 111. In particular, the perturbing protrusion 116′″ may contact the bottom of the bag 111 when the bag 111 is supported by the tray 140′ during operation of the bioreactor system 100′. As shown, the perturbing protrusion 116′″ may cause the bottom sheet 119 of the bag 111 to deform inwardly into the internal volume 117 normally provided by the inflated bag 111. In some embodiments, the perturbing protrusion 116′″ may be integrally formed with a remainder of the tray 140′. In some embodiments, the perturbing protrusion 116′″ may be separately formed and fixed to the top surface of the tray 140′. In this manner, the perturbing protrusion 116′″ may be formed as a part of or positioned on the tray 140′ and may cooperate with the bag 111 to have the same effect as the perturbing protrusion 116 described above.

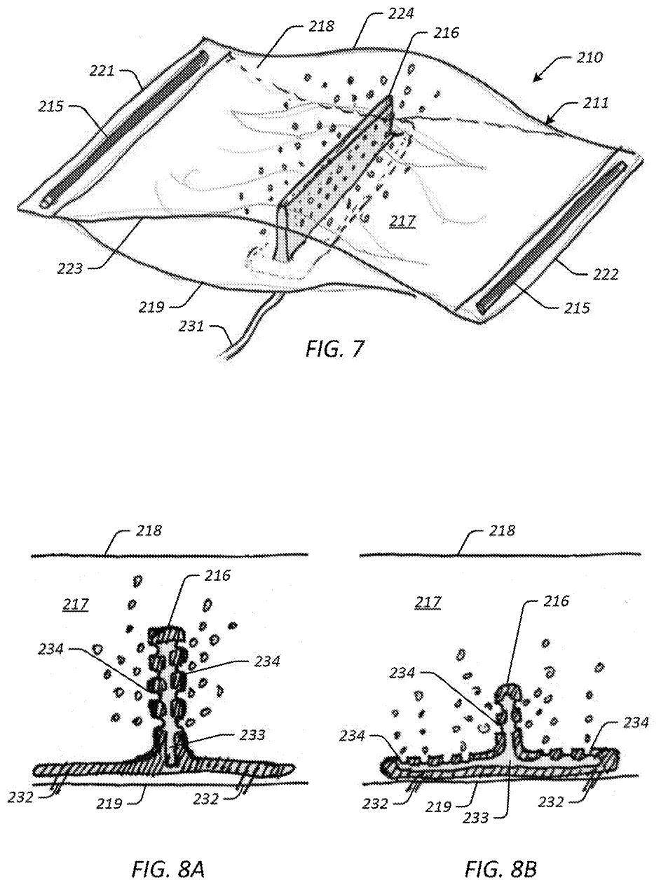

FIG. 7 illustrates a bioreactor 210 (which also may be referred to herein as a “bioreactor bag,” an “inflatable bioreactor,” or a “rockable bioreactor”) and components and features thereof according to one or more embodiments of the disclosure. The bioreactor 210 may be used with the bioreactor system 100 described above. Aside from the differences illustrated in FIG. 7 and described herein, the bioreactor 210 may be configured in a manner similar to the bioreactor 110 described above, including similar components and features. As shown in FIG. 7, the bioreactor 210 may include a bag 211, a pair of support rods 215, a perturbing protrusion 216, and an inlet port 231.

The bag 211 may be formed of a flexible sheet material and may be inflated during use of the bioreactor 210, such that the bag 211 has an internal volume 217 in which the culture medium 10 and cells can be aseptically cultured. The bag 211 may be formed by one or more sheets of flexible material, such as a flexible plastic material. As shown in FIG. 7, the bag 211 may include a top sheet 218 and a bottom sheet 219 of flexible material joined together, for example by heat sealing, to form the bag 211. In some embodiments, the bag 211 may have a first end edge 221 and a second end edge 222 positioned opposite one another, and a first side edge 223 and a second side edge 224 positioned opposite one another. As shown, the support rods 215 may be attached to the bag 211 and positioned at or near the respective ends of the bag 211. The support rods 215 may facilitate removable attachment of the bioreactor 210 to the tray 140 and maintaining the position and orientation of the bioreactor 210 relative to the tray 140 during operation of the bioreactor system 100.

As shown in FIG. 7, the perturbing protrusion 216 (which also may be referred to herein as a “turbulence protrusion,” a “sparging protrusion,” or a “turbulence rib”) may be positioned within the bag 211 at or adjacent the bottom of the bag 211. The perturbing protrusion 216 may be fixed to the bag 211. For example, the perturbing protrusion 216 may be attached to the bottom sheet 219 of the bag 211 by one or more heat seals 232, as shown in FIGS. 8A and 8B. The perturbing protrusion 216 may be configured to provide necessary oxygenation for initial low volume cultures by agitating small initial volumes of the cell culture medium 10 in a manner similar to the perturbing protrusion 116 described above. The perturbing protrusion 216 also may be configured to facilitate sparging of the cell culture medium 10 within the bag 211. As shown, the perturbing protrusion 216 may include an internal chamber 233 and a plurality of sparge holes 234 defined therein. The internal chamber 233 may be in fluid communication with the inlet port 231 for receiving a gas, such as oxygen, therefrom. During use of the bioreactor 210, the gas may pass through the internal chamber 233 and out of the sparge holes 234 into the cell culture medium 10 in the bag 211. In this manner, the perturbing protrusion 216 may provide improved oxygen transfer capacity, thereby expanding the operating range of the bioreactor 210 for applications requiring higher oxygen transfer rates. In some embodiments, the sparge holes 234 may be positioned on the vertical surfaces of the perturbing protrusion 216, as shown in FIG. 8A. In some embodiments, the sparge holes 234 may be positioned on the vertical surfaces and the horizontal surfaces of the perturbing protrusion 216, as shown in FIG. 8B. Various arrangements of the sparge holes 234 on the perturbing protrusion 216 may be used for different applications and performance benefits.

FIG. 7 illustrates a bioreactor 210 (which also may be referred to herein as a “bioreactor bag,” an “inflatable bioreactor,” a “rockable bioreactor,” or a “sparging bioreactor”) and components and features thereof according to one or more embodiments of the disclosure. The bioreactor 210 may be used with the bioreactor system 100 described above. Aside from the differences illustrated in FIGS. 7-8B and described herein, the bioreactor 210 may be configured in a manner similar to the bioreactor 110 described above, including similar components and features. As shown in FIG. 7, the bioreactor 210 may include a bag 211, a pair of support rods 215, a perturbing protrusion 216, and an inlet port 231.

The bag 211 may be formed of a flexible sheet material and may be inflated during use of the bioreactor 210, such that the bag 211 has an internal volume 217 in which the culture medium 10 and cells can be aseptically cultured. The bag 211 may be formed by one or more sheets of flexible material, such as a flexible plastic material. As shown in FIG. 7, the bag 211 may include a top sheet 218 and a bottom sheet 219 of flexible material joined together, for example by heat sealing, to form the bag 211. In some embodiments, the bag 211 may have a first end edge 221 and a second end edge 222 positioned opposite one another, and a first side edge 223 and a second side edge 224 positioned opposite one another. As shown, the support rods 215 may be attached to the bag 211 and positioned at or near the respective ends of the bag 211. The support rods 215 may facilitate removable attachment of the bioreactor 210 to the tray 140 and maintaining the position and orientation of the bioreactor 210 relative to the tray 140 during operation of the bioreactor system 100.

As shown in FIG. 7, the perturbing protrusion 216 (which also may be referred to herein as a “turbulence protrusion,” a “sparging protrusion,” or a “turbulence rib”) may be positioned within the bag 211 at or adjacent the bottom of the bag 211. The perturbing protrusion 216 may be fixed to the bag 211. For example, the perturbing protrusion 216 may be attached to the bottom sheet 219 of the bag 211 by one or more heat seals 232, as shown in FIGS. 8A and 8B. The perturbing protrusion 216 may be configured to provide necessary oxygenation for initial low volume cultures by agitating small initial volumes of the cell culture medium 10 in a manner similar to the perturbing protrusion 116 described above. The perturbing protrusion 216 also may be configured to facilitate sparging of the cell culture medium 10 within the bag 211. As shown, the perturbing protrusion 216 may include an internal chamber 233 and a plurality of sparge holes 234 defined therein. The internal chamber 233 may be in fluid communication with the inlet port 231 for receiving a gas, such as oxygen, therefrom. During use of the bioreactor 210, the gas may pass through the internal chamber 233 and out of the sparge holes 234 into the cell culture medium 10 in the bag 211. In this manner, the perturbing protrusion 216 may provide improved oxygen transfer capacity, thereby expanding the operating range of the bioreactor 210 for applications requiring higher oxygen transfer rates. In some embodiments, the sparge holes 234 may be positioned on the vertical surfaces of the perturbing protrusion 216, as shown in FIG. 8A. In some embodiments, the sparge holes 234 may be positioned on the vertical surfaces and the horizontal surfaces of the perturbing protrusion 216, as shown in FIG. 8B. Various arrangements of the sparge holes 234 on the perturbing protrusion 216 may be used for different applications and performance benefits.

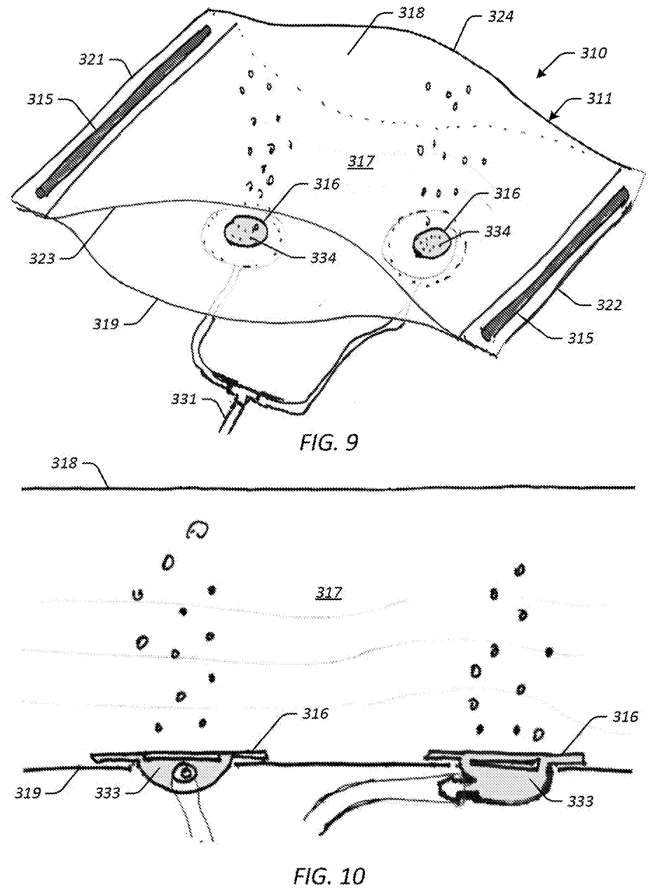

FIG. 9 illustrates a bioreactor 310 (which also may be referred to herein as a “bioreactor bag,” an “inflatable bioreactor,” a “rockable bioreactor,” or a “sparging bioreactor”) and components and features thereof according to one or more embodiments of the disclosure. The bioreactor 310 may be used with the bioreactor system 100 described above. Aside from the differences illustrated in FIGS. 9 and 10 and described herein, the bioreactor 310 may be configured in a manner similar to the bioreactor 110 described above, including similar components and features. As shown in FIG. 9, the bioreactor 310 may include a bag 311, a pair of support rods 315, a pair of sparge ports 316, and an inlet port 331.

The bag 311 may be formed of a flexible sheet material and may be inflated during use of the bioreactor 310, such that the bag 311 has an internal volume 317 in which the culture medium 10 and cells can be aseptically cultured. The bag 311 may be formed by one or more sheets of flexible material, such as a flexible plastic material. As shown in FIG. 9, the bag 311 may include a top sheet 318 and a bottom sheet 319 of flexible material joined together, for example by heat sealing, to form the bag 311. In some embodiments, the bag 311 may have a first end edge 321 and a second end edge 322 positioned opposite one another, and a first side edge 323 and a second side edge 324 positioned opposite one another. As shown, the support rods 315 may be attached to the bag 311 and positioned at or near the respective ends of the bag 311. The support rods 315 may facilitate removable attachment of the bioreactor 310 to the tray 140 and maintaining the position and orientation of the bioreactor 310 relative to the tray 140 during operation of the bioreactor system 100.

As shown in FIGS. 9 and 10, the sparge ports 316 may be positioned along the bottom of the bag 311 and in fluid communication with the internal volume 317 thereof. In some embodiments, as shown, each of the sparge ports 316 may include an internal portion that is positioned within the bag 311 and an external portion that is positioned outside of the bag, although other configurations of the sparge ports 316 may be used. The sparge ports 316 may be fixed to the bag 311. For example, the sparge ports 316 may be attached to the bottom sheet 319 of the bag 311 by one or more heat seals or other means of attachment. The sparge ports 316 may be configured to facilitate sparging of the cell culture medium 10 within the bag 311. As shown, each of the sparge ports 316 may include an internal chamber 333 and a plurality of sparge holes 334 defined therein. The internal chamber 333 may be in fluid communication with the inlet port 331 for receiving a gas, such as oxygen, therefrom. During use of the bioreactor 310, the gas may pass through the internal chamber 333 and out of the sparge holes 334 into the cell culture medium 10 in the bag 311. In this manner, the sparge ports 316 may provide improved oxygen transfer capacity, thereby expanding the operating range of the bioreactor 310 for applications requiring higher oxygen transfer rates. In some embodiments, as shown, internal portions of the sparge ports 316 may be formed as disc-shaped members having a substantially planar, horizontal top surface, and the sparge holes 34 may be positioned on the top surfaces of the sparge ports 316. Various arrangements of sparge ports 316 and the sparge holes 334 thereof may be used for different applications and performance benefits. Although the bioreactor 310 is illustrated in FIGS. 9 and 10 as having two sparge ports 316 spaced apart from one another in the direction of intended wave motion W, the bioreactor 310 may include any number of sparge ports 316 arranged in various configurations with respect to the bag 311.

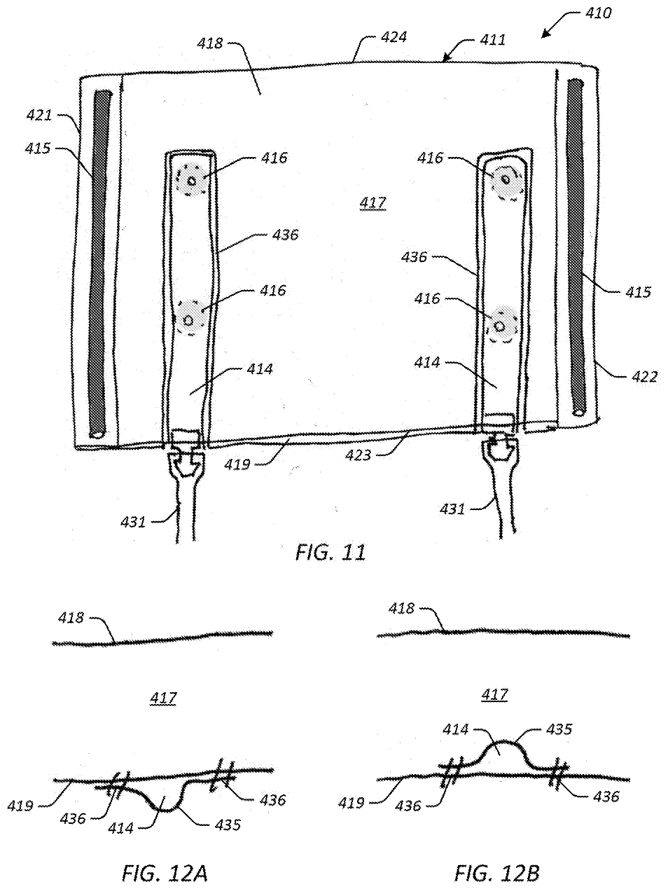

FIG. 11 illustrates a bioreactor 410 (which also may be referred to herein as a “bioreactor bag,” an “inflatable bioreactor,” a “rockable bioreactor,” or a “sparging bioreactor”) and components and features thereof according to one or more embodiments of the disclosure. The bioreactor 410 may be used with the bioreactor system 100 described above. Aside from the differences illustrated in FIGS. 11-12B and described herein, the bioreactor 410 may be configured in a manner similar to the bioreactor 110 described above, including similar components and features. As shown in FIG. 11, the bioreactor 410 may include a bag 411, a pair of rocking stabilization support rods 415, a plurality of sparge ports 416, a pair of sparge channels 414, and a pair of inlet ports 431.

The bag 411 may be formed of a flexible sheet material and may be inflated during use of the bioreactor 410, such that the bag 411 has an internal volume 417 in which the culture medium 10 and cells can be aseptically cultured. The bag 411 may be formed by one or more sheets of flexible material, such as a flexible plastic material. As shown in FIGS. 9-12A, the bag 411 may include a top sheet 418 and a bottom sheet 419 of flexible material joined together, for example by heat sealing, to form the bag 411. In some embodiments, the bag 411 may have a first end edge 421 and a second end edge 422 positioned opposite one another, and a first side edge 423 and a second side edge 424 positioned opposite one another. As shown, the support rods 415 may be attached to the bag 411 and positioned at or near the respective ends of the bag 411. The support rods 415 may facilitate removable attachment of the bioreactor 410 to the tray 140 and maintaining the position and orientation of the bioreactor 410 relative to the tray 140 during operation of the bioreactor system 100.

As shown in FIG. 11, the sparge ports 416 may be positioned along the bottom of the bag 411 and in fluid communication with the internal volume 417 thereof. In some embodiments, each of the sparge ports 416 may include an internal portion that is positioned within the bag 411 and an external portion that is positioned outside of the bag 411. In some embodiments, each of the sparge ports 416 may be positioned entirely within the bag 411 or entirely outside of the bag 411. The sparge ports 416 may be fixed to the bag 411. For example, the sparge ports 416 may be attached to the bottom sheet 419 of the bag 411 by one or more heat seals or other means of attachment. The sparge ports 416 may be configured to facilitate sparging of the cell culture medium 10 within the bag 411. Similar to the sparge ports 316 described above, each of the sparge ports 416 may include an internal chamber and a plurality of sparge holes defined therein.

As shown in FIGS. 11-12B, the sparge channels 414 may be positioned along the bottom of the bag 411 and in fluid communication with the internal volume 417 thereof via the sparge ports 416. In some embodiments, as shown, each of the sparge channels 414 may be formed by a sheet 435 of flexible material, such as a flexible plastics material, that is fixed to the bottom sheet 419 of the bag 411. For example, the sheets 435 may be attached to the bottom sheet 419 of the bag 411 by one or more heat seals 436 or other means of attachment. In some embodiments, as shown in FIG. 12A, the sheets 435 may be attached to the external surface of the bottom sheet 419, such that the sparge channels 414 are positioned outside of the bag 411. In some embodiments, as shown in FIG. 12B, the sheets 435 may be attached to the internal surface of the bottom sheet 419, such that the sparge channels 414 are positioned within the bag 411. As shown in FIG. 11, one or more of the sparge ports 416 may be positioned within each of the sparge channels 414. In other words, the each of the sheets 435 may surround one or more of the sparge ports 416. The sparge channels 414 may be in fluid communication with the respective inlet ports 431, as shown. In this manner, the sparge ports 416 may be in fluid communication with the inlet ports 431, via the sparge channels 414, for receiving a gas, such as oxygen, therefrom. During use of the bioreactor 410, the gas may pass through the sparge channels 414, through the sparge ports 416, and out of the sparge holes thereof into the cell culture medium 10 in the bag 411. In this manner, the sparge channels 414 and the sparge ports 416 may provide improved oxygen transfer capacity, thereby expanding the operating range of the bioreactor 410 for applications requiring higher oxygen transfer rates. As compared to the bioreactor 310 described above, the configuration of the sparge channels 414 of the bioreactor 410 may eliminate the need for tubing (i.e., portions of the inlet port 331 or intermediate tubing segments) along the bottom of the bioreactor 410. Various arrangements of the sparge channels 414, the sparge ports 416, and the sparge holes thereof may be used for different applications and performance benefits. Although the bioreactor 410 is illustrated in FIG. 11 as having two sparge channels 414 and four sparge ports 416 spaced apart from one another in the direction of intended wave motion W, the bioreactor 410 may include any number of sparge channels 414 and sparge ports 416 arranged in various configurations with respect to the bag 411. Further, in some embodiments, the sparge ports 416 may be omitted, and holes or perforations may be formed in the bottom sheet 419 of the bag 411 or the sheets 435 defining the sparge channels 414 (depending on whether the sparge channels 414 are positioned outside of or inside of the bag 411) to allow gas to pass directly from the sparge channels 414 into the internal volume 417 of the bag 411.

Many modifications of the embodiments of the present disclosure will come to mind to one skilled in the art to which the disclosure pertains upon having the benefit of the teachings presented herein through the foregoing descriptions and the associated drawings. Therefore, it is to be understood that the present invention is not to be limited to the specific embodiments disclosed and that modifications and other embodiments are intended to be included within the scope of the appended claims. Although specific terms are employed herein, they are used in a generic and descriptive sense only and not for purposes of limitation.

1. An inflatable bioreactor comprising one or more sheets joined to form a bag including a top sheet and a bottom sheet formed from the one or more sheets and being inflatable to provide an internal volume suitable for retaining a volume of culture liquid during flow of the culture liquid resulting from a rocking motion of the bag, the bioreactor further including one or more perturbing protrusions extending upwardly from the bottom sheet toward, but not as far as, the top sheet and extending transversely to, or obliquely to, a direction of wave motion of the culture liquid.

2. An inflatable bioreactor as claimed in claim 1, wherein the one or more perturbing protrusions is heat-sealed to the bottom sheet.

3. An inflatable bioreactor as claimed in claim 1, wherein the one or more perturbing protrusions extends linearly in a direction transverse to or oblique to the direction of wave motion.

4. An inflatable bioreactor as claimed in claim 1, wherein the bag comprises a first end edge and a second end edge positioned opposite one another, and a first side edge and a second side edge positioned opposite one another.

5. An inflatable bioreactor as claimed in claim 4, wherein the one or more perturbing protrusions is positioned centrally between the first end edge and the second end edge.

6. An inflatable bioreactor as claimed in claim 5, wherein the one or more perturbing protrusions has a vertical dimension that is equal to or less than about ¼ of a height of the fully inflated bag at a point above the one or more perturbing protrusions.

7. An inflatable bioreactor as claimed in claim 5, wherein the one or more perturbing protrusions has a horizontal dimension that is equal to or greater than about ½ of a distance between the first side edge and the second side edge.

8. An inflatable bioreactor as claimed in claim 4, wherein the one or more perturbing protrusions includes a first perturbing protrusion and a second perturbing protrusion spaced apart from one another in a direction from the first side edge to the second side edge to define a gap therebetween.

9. An inflatable bioreactor as claimed in claim 4, wherein the one or more perturbing protrusions includes a first perturbing protrusion and a second perturbing protrusion spaced apart from one another in a direction from the first end edge to the second end edge.

10. An inflatable bioreactor as claimed in claim 1, wherein the one or more perturbing protrusions has an inverted T-shape.

11. An inflatable bioreactor as claimed in claim 1, wherein the one or more perturbing protrusions includes an internal chamber and a plurality of sparge holes for directing a gas into the internal volume of the bag.

12. An inflatable bioreactor as claimed in claim 11, wherein the sparge holes are positioned on vertical surfaces of the one or more perturbing protrusions.

13. An inflatable bioreactor as claimed in claim 11, wherein the sparge holes are positioned on horizontal surfaces of the one or more perturbing protrusions.

14. A bioreactor system comprising a tray suitable for supporting an inflatable bioreactor in a rocking motion, the bioreactor comprising one or more sheets joined to form a bag including a top sheet and a bottom sheet formed from the one or more sheets and being inflatable to provide an internal volume suitable for retaining a volume of culture liquid during flow of the culture liquid resulting from the rocking motion of the bag, the system further including one or more perturbing protrusions extending upwardly from the bottom sheet toward, but not as far as, the top sheet and extending transversely to, or obliquely to, a direction of wave motion of the culture liquid.

15. A bioreactor system as claimed in claim 14, wherein the one or more protrusions includes one or more upward extensions of the tray.

16. An inflatable bioreactor comprising one or more sheets joined to form a bag including a top sheet and a bottom sheet formed from the one or more sheets and being inflatable to provide an internal volume suitable for retaining a volume of culture liquid during flow of the culture liquid resulting from a rocking motion of the bag, the bioreactor further including one or more sparge ports positioned at least partially within the bag and attached to the bottom sheet, the one or more sparge ports in fluid communication with the internal volume and configured for delivering a gas thereto.

17. An inflatable bioreactor as claimed in claim 16, further comprising one or more inlet ports in fluid communication with the one or more sparge ports and configured for delivering the gas thereto.

18. An inflatable bioreactor as claimed in claim 16, further comprising one or more sparge channels in fluid communication with the one or more sparge ports and configured for delivering the gas thereto, the one or more sparge channels defined by one or more sheets attached to the bottom sheet.

19. An inflatable bioreactor as claimed in claim 18, wherein the one or more sparge channels is positioned outside of the bag.

20. An inflatable bioreactor as claimed in claim 18, wherein the one or more sparge channels is positioned within the bag.
